(19) United States
(12) Patent Application Publication
(10) Pub. No.: US 2019/0038263 A1
(43) Pub. Date: Feb. 7, 2019

(54) EXUDATE SAMPLING DEVICE

(71) Applicant: DOLHAY KFT., Budapest (HU)

(72) Inventor: Balazs Imre DOLHAY, Budapest (HU)

(21) Appl. No.: 15/755,639

(22) PCT Filed: Aug. 30, 2016
(86) PCT No.: PCT/HU2016/050037

§ 371 Date: Feb. 27, 2018

(30) Foreign Application Priority Data
Aug. 31, 2015 (HU) P1600196

(51) Int. Cl. A61B 10/00; A61B 10/02

(52) U.S. Cl. CPC A61B 10/0045; A61B 10/0291; A61B 2010/0216; A61B 2010/0074; A61B 2010/0006

(57) ABSTRACT

This invention relates to a sampling device for exudates, having a handle and a sampling member connected to the handle, wherein the handle is formed as a length of tube (1) open at both its ends (4,5), and the sampling member is an elastic sampling bag closed at one end (21) and attached air tightly to the distal end (5) of the tube (1) by its other end, and an elastic activating bag (3) is attached air tightly to the proximal end (4) of the tube (1).

The invention relates to a sampling device for exudates, having a handle and a sampling member connected to the handle.

Since the inflammation or tumor arising on the shell portion of the human uterus (portio vaginalis uteri) and on the wall of the vagina can be detected by a sample obtained from mucus of the vagina, sampling instruments allowing sample taking from the vaginal mucosa already known in the prior art, or even drugs can be delivered to the vagina by means of these devices.

Patent publication no. US2015088032 (A1). for example, discloses an endometrial sampler having a handle with distal end and a longitudinal axis, and an elongated rod extending from the handle distal end along the longitudinal axis, and wherein the rod has an outer surface, front and rear ends and the rear end is fixed to a distal end portion of the handle. A reciprocating actuator is connected to a portion of the handle and of a sheath. The sheath can envelop a part of the rod, and is fixed to the rod near the front end of the rod, and it is fixed to the first end of the actuating element. The distal end of the sheath is slit, and a portion of the rod lying beneath the distal end portion of the exterior of the sheath forms a sample collecting cavity. The device can be put in her body cavity by the user itself, even at home. The user sends the device once removed along with samples stored in the closed sheath in place of analysis in a protective packaging. A drawback of the device is that the sample can be taken only from internal wall of the vagina but not from the surface of the cervix. Another disadvantage is that sampling section of the device is quite complex and therefore relatively expensive and fragile, and laic application of the device is not possible.

Object of the invention is to provide an exudate sampling device, which allows the user to be able herself to take samples of the mucus of the cervix to be sent to the place of analysis. A further object to provide a device being as simple as possible, that is can be used with minimal anatomical knowledge, and not the complete sampling device containing the collected samples has to be sent in place of the analysis, but the sample taken can be separated from the device without the risk of contamination.

Above aims is achieved by a sampling device for exudates according to the invention, having a handle and a sampling member connected to the handle, the handle is formed as a length of tube open at both its ends, and the sampling member is an elastic sampling bag closed at one end and attached air tightly to the distal end of the tube by its other end, and an elastic activating bag is attached air tightly to the proximal end of the tube.

The sampling bag is attached preferably to the external surface of distal end of the tube.

The sampling bag is attached advantageously to the internal surface of the tube.

The activating bag is attached to the internal surface of the tube.

The activating bag is preferably attached to the external surface of the tube.

Sampling fibers are arranged on the surface of closed end of the sampling bag.

A pH sensitive hue changing reagent is arranged on the surface of closed end of the sampling bag.

A handgrip is fixed on the tube between said bags.

A handgrip is fixed on the tube, and at least one of said bags is fixed to the tube by means of said handgrip.

The sapling device is arranged in a sampling kit further containing at least a lockable sampling container.

The invention will be described in details by disclosing its preferred embodiments referring to the drawing as attached. In the drawings

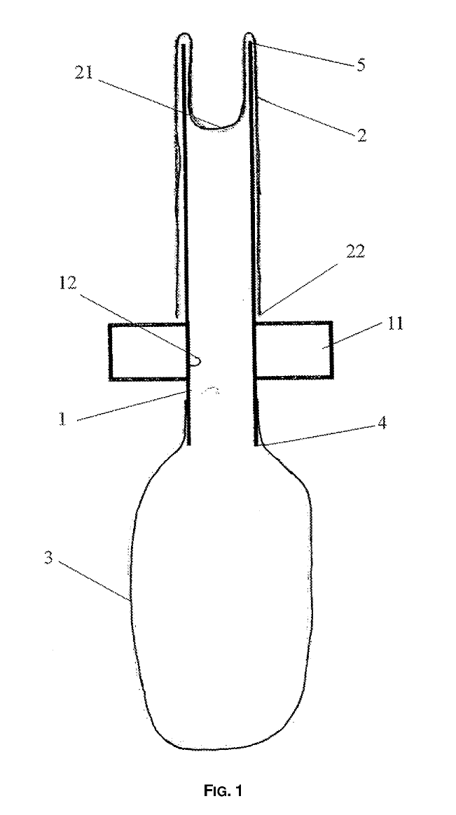

FIG. 1 shows a side elevational view of a preferred embodiment of exudate sampling device according to the invention in a normal position,

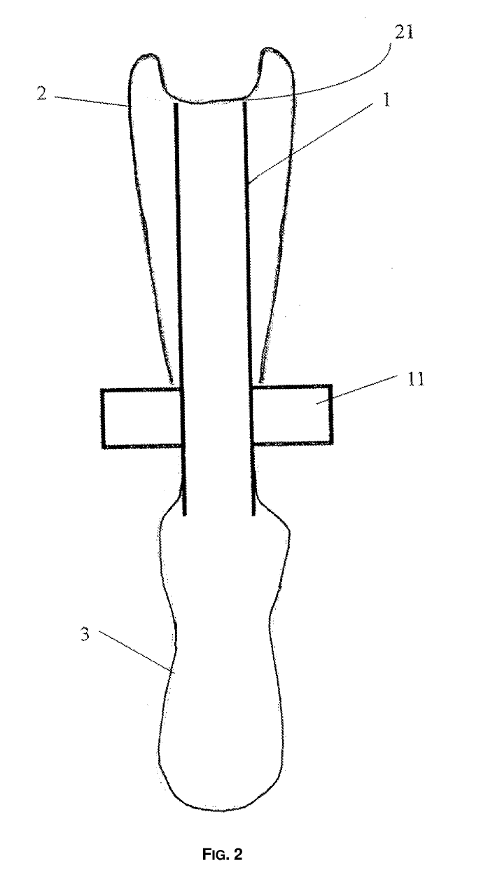

FIG. 2 shows a side elevational view of a preferred embodiment of exudate sampling device according to the invention in a sampling position, and in

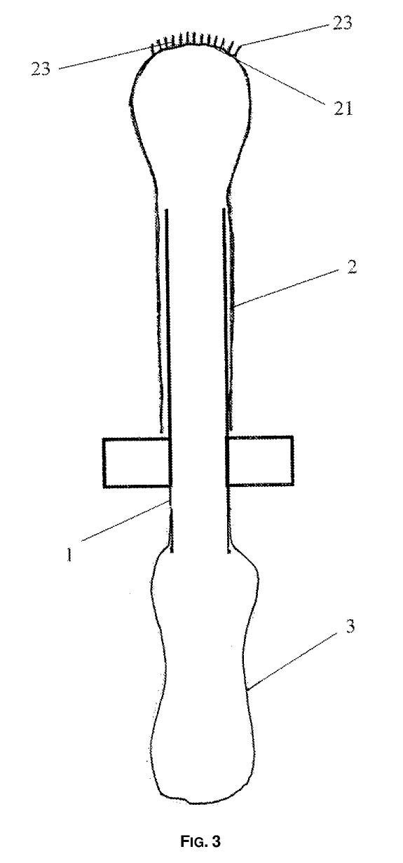

FIG. 3 the closed end of the sampling bag provided with sampling fibers is shown in inflated position.

FIG. 1 shows a preferred embodiment of exudate sampling device according to the invention in a normal position. The sampling device has a handle suitable for being grasped by the user and a sampling element connected airtight to the handle. In this embodiment, the handle is a length of pipe 1 open at both its ends 4,5, while the sampling element is a flexible sampling bag 2 closed at one end 21, and the other end 22 is fixed air tightly to the distal end 5 of the pipe 1 by pulling it over the outer surface of distal end 5 of the pipe 1 in the illustrated embodiment. The sampling bag 2 is responsible to gather exudates from the mucosal surface surrounding the cervix. A flexible activating bag 3 is fixed air tightly on the proximal end 4 of the pipe 1 by pulling it over and thus fixed on the outer surface of the pipe 1 in the illustrated embodiment. Fixing the sampling bag 2 and the activating bag 4 on the surface of the pipe 1 can be achieved by using an adhesive or by pressing on the pipe 1, wherein the pressing force is applied advantageously to the surface of the respective ends of the pipe 1 by the flexible material of the bags 2,3 in the illustrated embodiment, but in a case of fixing on the inner surface of the pipe 1 an inner grasping member (not shown) for the bag 2 and an inner or outer grasping member, e.g. clip ring, can be used for bag 3. Thus, the sampling bag 2, the actuating bag 3 and the pipe 1 form together a closed air-filled space in which the air pressure is preferably equal to ambient pressure. If the activating bag 3 of a sampling device thus formed is pressed e.g. manually, sampling bag 2 is inflated as shown in FIG. 3, since the interior of the two bags 2,3 and pipe 1 section form a single space isolated from the environment, and the material of the bags is a resilient one. Preferably, the actuator bag 3 is made from a little bit more rigid resilient material than the sampling bag 2, so that releasing the actuator bag 3 it is able to remove a portion of the air in the less rigid sampling bag 2, and thus pulling back the end 21 of the sampling bag 2 into the interior of the pipe 1. In a preferred embodiment shown in the figures a handle 11 is fixed to the pipe 1 between the bags 2, 3. In a further preferred embodiment at least one of the bags 2,3 is fixed to the pipe 1 section by means of the handle 11, that is the role of the above mentioned external clamping or grasping means may be achieved by the handle 1, too. In a simplest embodiment of the handle 11 it is a disc provided with a bore 12 in the middle, which disc is inserted into the pipe 1 section. The handle 11 is preferably constructed of a resilient material, and also serves for fixing the bags 2, 3 so that the open ends of the bags 2, 3 are arranged in the bore 12 between the handle 11 and the outer surface of the pipe section 1, such as bags 2, 3 are pressed to the outer surface of the pipe 1 by a clamping force exerted by the handle 11. In the case when outer clamping force is applied for fixing the sampling bag 2 to the outside surface of the pipe 1—applied e.g. by the handle 11—the sampling tube 2 can be easily replaced, and the sampling may be repeated either immediately. FIG. 2 shows the sampling device according to the invention in a sampling position, in which the sampling bag 2 is positioned around the cervix, not shown in Figure, affecting its shape. This situation can be reached by the user in such a way, that she has to lie in a semi-recumbent, relaxed position and grasping only the handle 11—or the pipe section 1 in absence of handle 11—introduces the sampling device according to the invention with the sampling bag 2 forward into the vagina until it the reaches the cervix. Subsequently, grasping the actuator bag 3 in such a way, that her little finger is near the vagina. While compressing the actuating bag 3 and pressing air from bag 3 over the pipe section 1 slowly into the sampling bag 2, she rotates the sampling device around its axis so that her thumb points twice her left and twice her right knee. Meanwhile, the end 21 of the sampling bag 2 of sampling device perfectly fitting to the cervix in a position as shown in FIG. 2 collects exudates from the surface of the cervix.

Subsequently, the user releases the actuator bag 3 elastic force of which sucks the air back from the sampling bag 2, and its end 21 is forced back into the interior of the pipe section 1, as shown in FIG. 1. The user pulls out the sampling device slowly from the vagina by grabbing the handle 11.

After sampling, the user squeezes the actuator bag 3 of the device removed from the vagina again, so that the end 21 of sampling bag 21 of the sampling device emerges from the interior of the pipe 1 as shown in FIG. 3. In an embodiment shown in FIG. 3 closed end 21 of sampling bag 2 is preferably provided with sampling fibers 23 that enhance the efficiency of the sampling. Sampling device according to the invention the sampling is provided preferably in a kit, which has at least a sterile lockable sampling container not shown in the Figures. The user cuts the ends 21 of the sampling bag 2 being in a state as shown in FIG. 3 e.g. by means of a scissor, so that the end 21 cut should be fallen into the sampling container, and then the container properly sealed could be transmitted in place of analysis for example by mail.

A pH sensitive hue-changing reagent can be arranged on the surface of closed end 21 of the sampling bag 2 between the sampling fibers 23. The color change shows any surface with abnormal vaginal exudates, where abnormal shaped elements can be more safely detected by the analytical laboratory.

The most significant advantage of the sampling device according to the invention over the prior art solutions is that it allows the user himself to be able to take samples from the mucus of the cervical canal and the surface of the portio vaginalis uteri, from which most frequent illnesses of lower genital tract start to spread. Exudates predicting abnormal processes of uterine cavity, ovary and stomach are also collected by this space. There can be found ovules (not) fertilized or trophoblast cells holding the chromosome of fetus. Another advantage is that sampling fibers 23 of the sampling bag 2 are tight-drawn, while the bag 2 is forced back into the interior of the pipe 1 and hence the sample will not be lost or contaminated by exudates of other surfaces upon removal of the device from the vagina. The sampling device with sampling bag 2 according to the invention functioning as tinned exudate may reach any automatic rapid-response laboratory installed anywhere by post as well. A further advantage of the sampling device according to the invention is that it has a simple construction, its use does not disturb the sense of decency, and in the embodiment comprising a sampling bag 3 fixed by clamping force the sampling process can be repeated at will by replacing the sampling bag 3, and time can be saved.

1-9. (canceled)

10. A sampling device for exudates, comprising a handle and a sampling member connected to the handle, the handle is formed as a length of tube open at both its ends, and the sampling member is an elastic sampling bag closed at one end and attached air tightly to the distal end of the tube by its other end, and an elastic activating bag is attached air tightly to the proximal end of the tube, characterized in that a pH sensitive hue changing reagent is arranged on the surface of closed end of the sampling bag.

11. The sampling device for exudates according to claim 10, wherein the sampling bag is attached to an external surface of distal end of the tube.

12. The sampling device for exudates according to claim 10, wherein the sampling bag is attached to an internal surface of the tube.

13. The sampling device for exudates according to claim 10, wherein the activating bag is attached to an internal surface of the tube.

14. The sampling device for exudates according to claim 10, wherein the activating bag is attached to an external surface of the tube.

15. The sampling device for exudates according to claim 10, wherein sampling fibers are arranged on the surface of closed end of the sampling bag.

16. The sampling device for exudates according to claim 15, wherein a handgrip is fixed on the tube between said bags.

17. The sampling device for exudates according to claim 15, wherein a handgrip is fixed on the tube, and at least one of said bags is fixed to the tube by means of said handgrip.

18. The sampling device for exudates according to claim 10, wherein the sampling device is arranged in a sampling kit further containing at least a lockable sampling container.
